(19) United States
(12) Plant Patent Application Publication
(10) Pub. No.: US 2014/0366231 P1
(43) Pub. Date: Dec. 11, 2014

(54) Gala schnico red

(50) ;

(71) Applicant: Schniga GmbH, Bozen (IT)

(72) Inventors: Andreas Gruber-Genetti, Lana/Vollan (IT); Thomas Braun, Girlan (IT); Walter Malleier, Lana/Vollan (IT)

(21) Appl. No.: 14/120,597

(22) Filed: Jun. 6, 2014

(30) Foreign Application Priority Data
Jun. 6, 2013 (EP) 2013/1524

(51) Int. Cl. A01H 5/00

(52) U.S. Cl. PLT/162

(57) ABSTRACT

Malus domestica Borkh ‘Gala Schnico red’ variety is distinguished by a unique combination of characteristics including an intensive red overcolor covering 100% of the fruit skin and a 30% higher number of lenticels per unit area on the fruit skin.

### RELATED APPLICATION INFORMATION

This application claims priority under 35 U.S. §119(a) to European Union Community Plant Variety Breeder's Rights Application No. 2013/1524, filed Jun. 6, 2013; the disclosure of which is incorporated herein by reference in its entirety.

### LATIN NAME OF THE GENUS AND SPECIES

The Latin name of the genus and species of the plant variety disclosed herein is Malus domestica Borkh.

### VARIETY DENOMINATION

The inventive cultivar of Malus domestica Borkh disclosed herein has been given the varietal denomination ‘Gala Schnico red.’

### BACKGROUND OF THE INVENTION

The present invention relates to a new and distinct Gala variety of apple trees named ‘Gala Schnico red.’ The new variety was discovered as a mutation on a Gala plant cultivar ‘Schnitzer Schniga’ in a cultivated field at 30904 Nals (BZ) Aniage Knoll, Italy.

‘Gala Schnico red’ has been asexually propagated by grafting and all the trees of ‘Gala Schnico red’ have been observed to remain true to type over successive asexually propagated generations and maintain their unique characteristics.

### SUMMARY OF THE INVENTION

The new variety ‘Gala Schnico red’ is distinguished from ‘Gala Schnico’, a comparator variety, by the following unique characteristics of an intensive red over colour covering 100% of the fruit skin as compared ‘Gala Schnico,’ which has an intensive red over colour covering only 80-90% of the fruit skin. In addition, ‘Gala Schnico red’ has a 30% higher number of lenticels per unit area on the fruit skin as compared to ‘Gala Schnico’.

Asexual reproduction of this new variety by grafting onto rootstock shows that the foregoing and all other characteristics and distinctions remain true to type over successive asexually propagated generations.

### BRIEF DESCRIPTION OF THE DRAWINGS

The colours of these illustrations may vary with lighting conditions and, therefore, colour characteristics of this new variety should be determined with reference to the observations described herein, rather than from these illustrations alone.

FIG. 1 shows a fruit of ‘Gala Schnico red.’

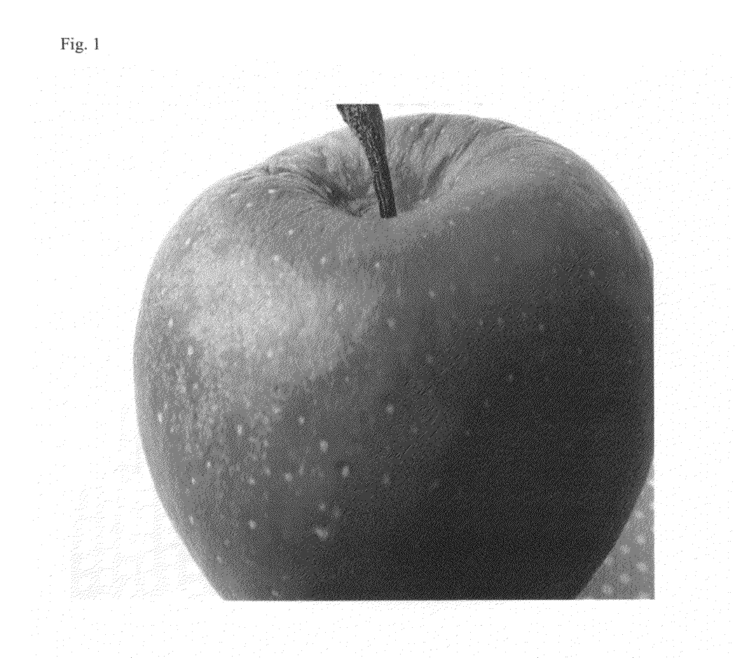

FIG. 2 provides a closer view of the fruit of ‘Gala Schnico red’ showing the lenticels.

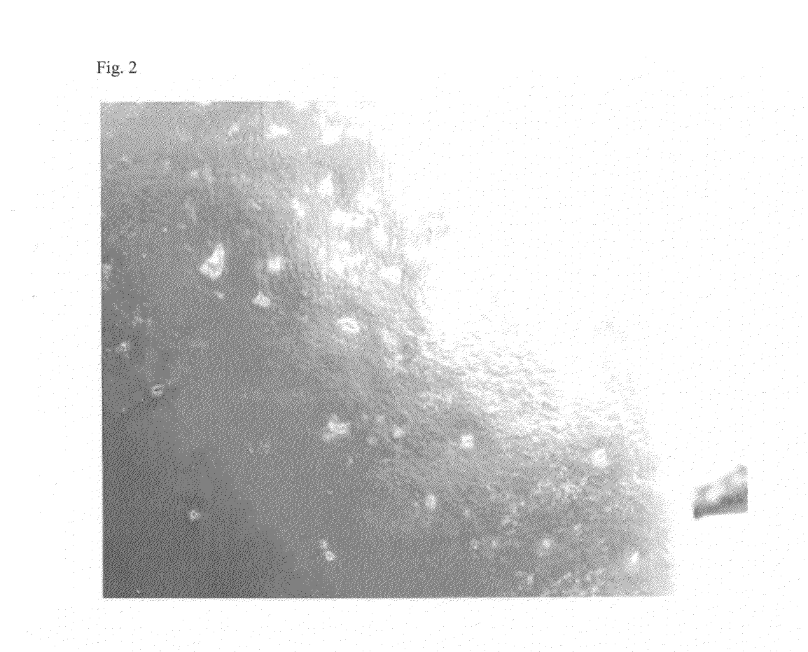

FIGS. 3 shows the flowers of ‘Gala Schnico red.’

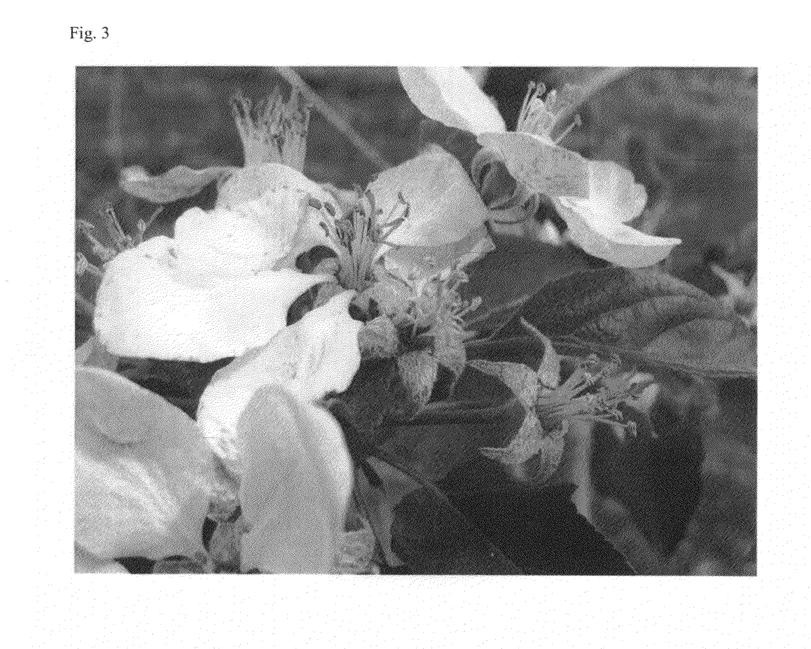

### DETAILED BOTANICAL DESCRIPTION

The following is a detailed description of the ‘Gala Schnico red’ variety.

Certain characteristics of this variety may change with changing environmental conditions (e.g., light, temperature, moisture), nutrient availability, or other factors. Quantified measurements are expressed as an average of measurements taken from a number of individual plants of the new variety. The measurements of any individual plant, or any group of plants, of the new variety may vary from the stated average. Colour descriptions and other terminology are used in accordance with their ordinary dictionary descriptions, unless the context clearly indicates otherwise. Colour designations are made with reference to The Royal Horticultural Society (R.H.S.) Colour Chart.
Parentage: ‘Gala Schnico red’ variety originated as a mutation of the Gala cultivar ‘Schnitzer Schniga’ identified in a cultivated field in Aniage Knoll, Italy.
Tree: the trees described here are three years old derived from the vegetatively propagated ‘Gala Schnico red’ mutation and grown on rootstock.
Tree type: ramified; Habit: spreading.
Time of beginning of flowering: medium; comparable to the apple varieties ‘Cox's Orange Pippin’ and ‘Jonagold.’
Fruit: Relative area of overall colour is very large and is comparable to the apple variety ‘Red Jonaprince.’ The hue of over colour with bloom removed is purple red, which is comparable to the apple varieties ‘Red Jonaprince’ and ‘Spartan.’ The pattern of over colour is solid flush with weakly defined stripes similar to the apple variety ‘Obro Gala.’
Pollination varieties: Red Delicious, Granny Smith and Golden Delicious.
Timing of eating maturity of fruit: Medium, comparable to the apple varieties ‘Elstar,’ ‘Gala’ and ‘Honeycrisp.’
Disease and insect resistance: No significant disease or insect pests have been observed.

What is claimed is:

1. A new and distinct cultivar of Malus domestica Borkh named ‘Gala Schnico red,’ substantially as illustrated and described herein.
